(19) United States
(12) Patent Application Publication
(10) Pub. No.: US 2012/0165260 A2
(43) Pub. Date: Jun. 28, 2012

(54) CRYSTALLINE EZATIOSTAT HYDROCHLORIDE ANSOLVATE

(75) Inventors: STEPHAN PARENT, WEST LAFAYETTE, IN (US); STEVEN SCHOW, REDWOOD CITY, CA (US)

(73) Assignee: TELIK, INC., PALO ALTO, CA (US)

(21) Appl. No.: 13/041,136

(22) Filed: Mar. 4, 2011

Related U.S. Application Data
(60) Provisional application No. 61/352,377, filed on Jun. 7, 2010.
(60) Provisional application No. 61/460,745, filed on Sep. 10, 2010.

(51) Int. Cl. A61K 38/06; A61P 35/02; A61P 35/00; C07K 5/093; A61P 7/00

(52) U.S. Cl. 514/13.5; 530/331; 514/19.6; 514/19.3

(57) ABSTRACT

Crystalline ezatiostat hydrochloride ansolvate form D is more stable and/or more soluble that various solvated crystalline polymorphic forms of ezatiostat hydrochloride.

### CROSS-REFERENCE TO RELATED APPLICATION

This application claims the benefit under 35 USC 119(e) of U.S. Provisional Application No. 61/352,377, filed Jun. 7, 2010, and U.S. Provisional Application No. 61/460,745, filed Sep. 10, 2010, each of which are hereby incorporated by reference in their entirety into this application.

### BACKGROUND

Ezatiostat hydrochloride is the hydrochloride acid addition salt of ezatiostat. Ezatiostat, also known as TLK199 or TER 199, is a compound of the formula:

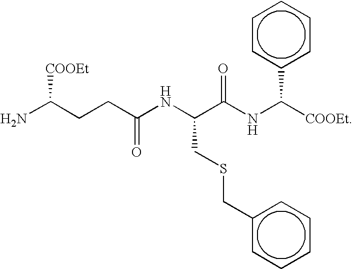

Ezatiostat has been shown to induce the differentiation of HL-60 promyelocytic leukemia cells in vitro, to potentiate the activity of cytotoxic agents both in vitro and in vivo, and to stimulate colony formation of all three lineages of hematopoietic progenitor cells in normal human peripheral blood. In preclinical testing, ezatiostat has been shown to increase white blood cell production in normal animals, as well as in animals in which white blood cells were depleted by treatment with cisplatin or fluorouracil. Similar effects may provide a new approach to treating myelodysplastic syndrome (MDS).

Many conditions, including MDS, a form of pre-leukemia in which the bone marrow produces insufficient levels of one or more of the three major blood elements (white blood cells, red blood cells, and platelets), are characterized by depleted bone marrow. Myelosuppression, which is characterized by a reduction in blood cell levels and in a reduction of new blood cell generation in the bone marrow, is also a common, toxic effect of many standard chemotherapeutic drugs.

Ezatiostat hydrochloride in a liposomal injectable formulation was studied in a clinical trial for the treatment of MDS, and results from this trial, reported by Raza et al., J. Hem. Onc., 2:20 (published online 13 May 2009), demonstrated that administration of TLK199 was well tolerated and resulted in multi-lineage hematologic improvement. Ezatiostat hydrochloride in a tablet formulation has been evaluated in a clinical trial for the treatment of MDS, as reported by Raza et al., Blood, 113:6533-6540 (prepublished online 27 Apr. 2009) and a single-patient report by Quddus et al., J. Hem. Onc., 3:16 (published online 23 Apr. 2010), and is currently being evaluated in clinical trials for the treatment of MDS and for severe chronic idiopathic neutropenia.

When used for treating humans, it is important that a crystalline therapeutic agent like ezatiostat hydrochloride retains its polymorphic and chemical stability, solubility, and other physicochemical properties over time and among various manufactured batches of the agent. If the physicochemical properties vary with time and among batches, the administration of a therapeutically effective dose becomes problematic and may lead to toxic side effects or to ineffective therapy, particularly if a given polymorph decomposes prior to use, to a less active, inactive, or toxic compound. Therefore, it is important to choose a form of the crystalline agent that is stable, is manufactured reproducibly, and has physicochemical properties favorable for its use as a therapeutic agent.

However, the art remains unable to predict which crystalline form of an agent will have a combination of the desired properties and will be suitable for human administration, and how to make the agent in such a crystalline form.

### SUMMARY

It has now been discovered that ezatiostat salts and, in particular, the hydrochloride salt, can be formed as a crystalline ansolvate, referred to here as form D. Surprisingly, this ansolvate demonstrates superior stability and other physicochemical properties compared to the solvate crystalline forms A, B, C, E, and F. Accordingly, in one aspect, this invention provides for crystalline ezatiostat ansolvate salt and, in particular, the hydrochloride salt (crystalline form D). In one embodiment, the crystalline ezatiostat hydrochloride ansolvate does not undergo polymorphic transformation. In another embodiment, the crystalline ezatiostat hydrochloride ansolvate is characterized by an endothermic peak at (177±2)° C. as measured by differential scanning calorimetry. In another embodiment, the crystalline ezatiostat hydrochloride ansolvate is characterized by the substantial absence of thermal events at temperatures below the endothermic peak at (177±2)° C. as measured by differential scanning calorimetry. In another embodiment, the crystalline ezatiostat hydrochloride ansolvate is characterized by an X-ray powder diffraction peak (Cu Kα radiation) at (2.7±0.2) °2θ. In another embodiment, the crystalline ezatiostat hydrochloride ansolvate is characterized by an X-ray powder diffraction peak (Cu Kα radiation) at (6.3±0.2) °2θ. In another embodiment, the crystalline ezatiostat hydrochloride ansolvate is characterized by an X-ray powder diffraction pattern (Cu Kα radiation) substantially similar to that of FIG. 6 or FIG. 7. In another embodiment, the crystalline ezatiostat hydrochloride ansolvate is characterized by a solid-state ¹³C nuclear magnetic resonance spectrum substantially similar to that of FIG. 8. In another embodiment, the crystalline ezatiostat hydrochloride ansolvate is characterized by at least two X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ). In another embodiment, the crystalline ezatiostat hydrochloride ansolvate is characterized by at least three X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ). In another embodiment, the crystalline ezatiostat hydrochloride ansolvate is characterized by at least one X-ray powder diffraction peak (Cu Kα radiation) selected from 2.7°, 6.3°, 7.3°, 8.2°, 8.4°, 9.6°, 11.0°, and 12.7 °2θ (each ±0.2 °2θ). In another embodiment, the crystalline ezatiostat hydrochloride is characterized by at least two X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ). In another embodiment, the crystalline ezatiostat hydrochloride ansolvate is characterized by at least three X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ).

In one of its composition embodiments, this invention provides a composition comprising the crystalline ezatiostat hydrochloride ansolvate. In one embodiment, the composition shows an aqueous solubility of at least about 5 mg/mL to about 8 mg/mL. In another embodiment, the crystalline ezatiostat hydrochloride ansolvate or the composition, when exposed to about 60% relative humidity at about 25° C. for about 6 months in the presence of a desiccant, does not show substantial formation of an impurity.

In another of its composition embodiments, this invention provides for a pharmaceutical composition comprising a pharmaceutically acceptable excipient and crystalline ezatiostat hydrochloride ansolvate.

In one of its method embodiments, this invention provides a method of preparing the solid crystalline ansolvate form D.

In another of its method embodiments, this invention provides a method of storing crystalline ezatiostat hydrochloride ansolvate such that the morphology of form D remains stable over its shelf-life and, indeed, for prolonged periods of time. In one aspect of this method, the crystalline ezatiostat hydrochloride ansolvate in an anhydrous environment (e.g., by using desiccants or vacuum conditions to maintain an anhydrous environment).

In still another of its method embodiments, there are provided methods for inducing differentiation of HL-60 promyelocytic leukemia cells in vitro, to potentiate the activity of cytotoxic agents both in vitro and in vivo, and/or to stimulate colony formation of all three lineages of hematopoietic progenitor cells in normal human peripheral blood.

In yet another of its method embodiments, there are provided methods of treating myelodysplastic syndrome, severe chronic idiopathic neutropenia, leukemia or other cancers and conditions that involve cytopenia, chemotherapy induced neutropenia, or thrombocytopenia comprising administering a therapeutically effective amount of crystalline ezatiostat hydrochloride ansolvate (form D) provided herein, or a composition comprising the ansolvate form D to a patient in need of such treatment.

In all of such treatments, the dosing of crystalline ezatiostat hydrochloride ansolvate to the treated patient is already disclosed in the art.

### BRIEF DESCRIPTION OF THE DRAWINGS

FIG. 1 is a DSC pattern of ezatiostat hydrochloride monohydrate form A.

FIG. 2 is an XRPD pattern of ezatiostat hydrochloride monohydrate form A.

FIG. 3 is a high-resolution XRPD pattern of ezatiostat hydrochloride monohydrate form A.

FIG. 4 is an SS-NMR spectrum of ezatiostat hydrochloride monohydrate form A.

FIG. 5 is a DSC pattern of crystalline ezatiostat hydrochloride ansolvate form D.

FIG. 6 is an XRPD pattern of crystalline ezatiostat hydrochloride ansolvate form D.

FIG. 7 is a high-resolution XRPD pattern of crystalline ezatiostat hydrochloride ansolvate form D.

FIG. 8 is an SS-NMR spectrum of crystalline ezatiostat hydrochloride ansolvate form D.

FIG. 9 is a comparative XRPD pattern of crystalline ezatiostat hydrochloride polymorphic forms A-F.

FIG. 10 is a comparative DSC pattern of crystalline ezatiostat hydrochloride polymorphic forms A, D, and E.

FIG. 11 is an SS-NMR spectrum of crystalline ezatiostat hydrochloride form E.

### DETAILED DESCRIPTION

As noted above, this invention is directed, in part, to a stable crystalline ansolvate of ezatiostat salts and, in particular, the hydrochloride salt. However, prior to discussing this invention in further detail, the following terms will be defined.

### DEFINITIONS

As used herein, the following terms have the following meanings.

The singular forms “a,” “an,” and “the” and the like include plural referents unless the context clearly dictates otherwise. Thus, for example, reference to “a compound” includes both a single compound and a plurality of different compounds.

The term “about” when used before a numerical designation, e.g., temperature, time, amount, and concentration, including a range, indicates approximations which may vary by ±10%, ±5% or ±1%.

“Administration” refers to introducing an agent into a patient. A therapeutic amount can be administered, which can be determined by the treating physician or the like. An oral route of administration is preferred. The related terms and phrases administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient. In any event, administration entails delivery to the patient of the drug.

The “crystalline ansolvate” of ezatiostat hydrochloride is a crystalline solid form of ezatiostat hydrochloride, such as, e.g., the crystalline form D. The form D crystal lattice is substantially free of solvents of crystallization. However, any solvent present is not included in the crystal lattice and is randomly distributed outside the crystal lattice. Therefore, form D crystals in bulk may contain, outside the crystal lattice, small amounts of one or more solvents, such as the solvents used in its synthesis or crystallization. As used above, “substantially free of” and “small amounts,” refers to the presence of solvents preferably less that 10,000 parts per million (ppm), or more preferably, less than 500 ppm.

“Characterization” refers to obtaining data which may be used to identify a solid form of a compound, for example, to identify whether the solid form is amorphous or crystalline and whether it is unsolvated or solvated. The process by which solid forms are characterized involves analyzing data collected on the polymorphic forms so as to allow one of ordinary skill in the art to distinguish one solid form from other solid forms containing the same material. Chemical identity of solid forms can often be determined with solution-state techniques such as ¹³C NMR or ¹H NMR. While these may help identify a material, and a solvent molecule for a solvate, such solution-state techniques themselves may not provide information about the solid state. There are, however, solid-state analytical techniques that can be used to provide information about solid-state structure and differentiate among polymorphic solid forms, such as single crystal X-ray diffraction, X-ray powder diffraction (XRPD), solid state nuclear magnetic resonance (SS-NMR), and infrared and Raman spectroscopy, and thermal techniques such as differential scanning calorimetry (DSC), thermogravimetry (TG), melting point, and hot stage microscopy.

To “characterize” a solid form of a compound, one may, for example, collect XRPD data on solid forms of the compound and compare the XRPD peaks of the forms. For example, when only two solid forms, I and II, are compared and the form I pattern shows a peak at an angle where no peaks appear in the form II pattern, then that peak, for that compound, distinguishes form I from form II and further acts to characterize form I. The collection of peaks which distinguish form I from the other known forms is a collection of peaks which may be used to characterize form I. Those of ordinary skill in the art will recognize that there are often multiple ways, including multiple ways using the same analytical technique, to characterize solid forms. Additional peaks could also be used, but are not necessary, to characterize the form up to and including an entire diffraction pattern. Although all the peaks within an entire XRPD pattern may be used to characterize such a form, a subset of that data may, and typically is, used to characterize the form.

An XRPD pattern is an x-y graph with diffraction angle (typically °2θ) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, it is usual to assign a variability of ±0.2° 2θ to diffraction angles in XRPD patterns.

X-ray powder diffraction (XRPD) analyses were performed on a Shimadzu XRD-6000 X-ray powder diffractometer using Cu Kα radiation from a long fine focus X-ray tube, operated at 40 kV, 40 mA. The divergence and scattering slits were set at 1° and the receiving slit was set at 0.15 mm. Diffracted radiation was detected by a NaI scintillation detector. A θ-2θ continuous scan at 3°/min (0.4 sec/0.02° step) from 2.5°-40° 2θ was used. A silicon standard was analyzed to check alignment of the instrument. Data were collected and analyzed using XRD-6000 v. 4.1 software.

High-resolution XRPD analyses were also performed on a PANalytical X'Pert PRO PW3040 diffractometer, using Cu Kα radiation produced by an Optix long fine-focus tube (45 kV, 40 mA). An elliptically graded multilayer mirror was used to focus the X-rays through the specimen, which was sandwiched between 3 μm films, analyzed in transmission geometry, and rotated to optimize orientation statistics. A beam-stop and helium purge were used to minimize the air-scattering background; Soller slits (divergence slit, 0.5°; scattering slit 0.25°) were used for the incident and diffracted beams to minimize axial divergence. Diffraction patterns were collected using a scanning position-sensitive X'Celerator detector located 240 mm from the specimen, over a scan range of 1.01°-39° 2θ with a scan speed of 1.2°/min (step size 0.017° 2θ). A silicon standard was analyzed to check alignment of the instrument. Data were collected and analyzed using X'Pert PRO Data Collector v. 2.2b software. Indexing and Pawley refinement of the ezatiostat hydrochloride monohydrate XRPD pattern was performed using Match v.2.4.0 software (SSCI) and verified using ChekCell v. Nov. 1, 2004 (http://www.ccp14.ac.uk/tutorial/lmgp/). Indexing and Pawley refinement of the crystalline ezatiostat hydrochloride ansolvate XRPD pattern was performed using DASH v. 3.1 software (Cambridge Crystallographic Data Center).

Variable-temperature XRPD (VT-XRPD) analysis was performed on a Shimadzu XRD-6000 diffractometer equipped with an Anton Paar HTK 1200 high temperature stage. The sample was packed in a ceramic holder and analyzed from 2.5°-40° 2θ at 3°/min (0.4 sec/0.02° step). The temperature was held constant during each XRPD scan. Temperature calibration was performed using vanillin and sulfapyridine standards. A silicon standard was analyzed to check alignment of the instrument; data were collected and analyzed using XRD-6000 v.4.1 software.

Differential scanning calorimetry (DSC) analyses were performed on a TA Instruments Q100 or 2920 differential scanning calorimeter, which was calibrated using indium as the reference material. The sample was placed into a standard aluminum DSC pan with an uncrimped lid, and the weight accurately recorded. The sample cell was equilibrated at 25° C. and heated under a nitrogen purge at a rate of 10° C./minute to a final temperature of 250° C. The variability of DSC data is affected by sample preparation and particularly by heating rate.

Solid-state NMR (SS-NMR) ¹³C cross-polarization magic angle spinning (CP/MAS) analyses were performed at room temperature on a Varian^{UNITY}INOVA-400 spectrometer (Larmor frequencies: ¹³C=100.542 MHz, ¹H=399.800 MHz). The sample was packed into a 4 mm PENCIL type zirconia rotor and rotated at 12 kHz at the magic angle. The spectrum was acquired with phase modulated SPINAL-64 high power ¹H decoupling during the acquisition time using a ¹H pulse width of 2.2 μs (90°), a ramped amplitude cross polarization contact time of 2 ms, a 30 ms acquisition time, a 5 second delay between scans, a spectral width of 45 KHz with 2700 data points, and 200 co-added scans. The free induction decay (FID) was processed using Varian VNMR 6.1C software with 32768 points and an exponential line broadening factor of 10 Hz to improve the signal-to-noise ratio. The first three data points of the FID were back predicted using the VNMR linear prediction algorithm to produce a flat baseline. The chemical shifts of the spectral peaks were externally referenced to the carbonyl carbon resonance of glycine at 176.5 ppm. The variability of SS-NMR peaks in this experiment is considered to be ±0.2 ppm.

Karl Fischer analyses for water determination were performed on a Mettler Toledo DL39 Karl Fischer titrator. About 10-15 mg of sample was placed in the KF titration vessel containing approximately 100 mL of Hydranal®-Coulomat AD reagent and mixed for 60 seconds to ensure dissolution. The dissolved sample was then titrated by means of a generator electrode which produces iodine by electrochemical oxidation.

Thermogravimetric (TG-IR) analyses were performed on a TA Instruments model 2050 thermogravimetric (TG) analyzer interfaced to a Thermo Nicolet Magna® 560 Fourier transform infrared (FT-IR) spectrophotometer equipped with a Ever-Glo mid/far IR source, a potassium bromide beamsplitter, and a deuterated triglycine sulfate detector. The instrument was operated under a flow of helium at 90 mL/min (purge) and 10 mL/min (balance). The sample was placed in a platinum sample pan, inserted into the TG furnace, accurately weighed by the instrument, and heated from ambient at a rate of 20° C./min. The TG instrument was started first, immediately followed by the FT-IR instrument. IR spectra were collected every 12.86 seconds; and each IR spectrum represents 32 co-added scans collected at a spectral resolution of 4 cm⁻¹. A background scan was collected before the beginning of the experiment. Wavelength calibration was performed using polystyrene. The TG calibration standards were nickel and Alumel™.

Hot stage microscopy analysis was performed on a Linkam FTIR 600 hot stage mounted on a Leica DM LP microscope. Samples were observed using a 20× objective with cross polarizers and lambda compensator. A coverslip was then placed over the sample. Each sample was visually observed as the stage was heated. Images were captured using a SPOT Insight™ color digital camera with SPOT Software v. 3.5.8. The hot stage was calibrated using USP melting point standards.

“Comprising” or “comprises” is intended to mean that the compositions and methods include the recited elements, but not exclude others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “does not undergo polymorphic transformation” refers to no observable polymorphic transformation of a crystalline form, when exposed to up to about 75% relative humidity at up to about 40° C. for up to about 6 months, when analyzed by XRPD or HPLC or another equivalently sensitive technique.

“Desiccant” refers to a substance that induces or sustains a state of dryness in its local vicinity in a moderately well-sealed container. Desiccants can absorb or adsorb water, or act by a combination of the two. Desiccants may also work by other principles, such as chemical bonding of water molecules. A pre-packaged desiccant may be used to remove excessive humidity that would degrade products. Non-limiting examples of desiccants include silica gel, calcium sulfate, calcium chloride, montmorillonite clay, and molecular sieves.

“Room temperature” refers to (22±5)° C.

“Storing” or “storage” refers to storing crystalline ezatiostat hydrochloride ansolvate form D or a composition including the form D such that no more than about 10%, more preferably no more than about 5%, still more preferably no more than about 3%, or most preferably no more than about 1% of the ansolvate form D undergoes transformation to another compound.

“Therapeutically effective amount” or “therapeutic amount” refers to an amount of a drug or an agent that when administered to a patient suffering from a condition, will have the intended therapeutic effect, e.g., alleviation, amelioration, palliation or elimination of one or more manifestations of the condition in the patient. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating myelodysplastic syndrome, refers to an amount of the agent that alleviates, ameliorates, palliates, or eliminates one or more manifestations of the myelodysplastic syndrome in the patient.

“Treatment”, “treating”, and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition. For purposes of this invention, beneficial or desired clinical results include, but are not limited to, multilineage hematologic improvement, decrease in the number of required blood transfusions, decrease in infections, decreased bleeding, and the like.

Identifying The Ansolvate Form D

A solid form screen was carried out on ezatiostat hydrochloride, starting with ezatiostat hydrochloride monohydrate form A, which was previously known. Both thermodynamic and kinetic crystallization techniques were employed. Once solid samples were harvested from crystallization attempts, they were examined under a microscope for birefringence and morphology. The solid samples were characterized by various techniques including those described above. A number of different crystallization techniques were used as set forth below.

Fast evaporation: solutions were prepared in various solvents and sonicated between aliquot additions to assist in dissolution. Once a mixture reached complete dissolution, as judged by visual observation, the solution was filtered through a 0.2 μm nylon filter. The filtered solution was allowed to evaporate at room temperature in an open vial, and the solids that formed were isolated by filtration and dried.

Slow evaporation: solutions were prepared as for the fast evaporation technique above, and the filtered solution was allowed to evaporate at room temperature in a vial covered with aluminum foil perforated with pinholes. The solids that formed were isolated by filtration and dried.

Slow cooling: saturated solutions were prepared in various solvents at elevated temperatures and filtered through a 0.2 μm nylon filter into an open vial while still warm. The vial was covered and allowed to cool slowly to room temperature, and the presence or absence of solids was noted. If there were no solids present, or if the amount of solids was judged too small for XRPD analysis, the vial was placed in a refrigerator overnight. Again, the presence or absence of solids was noted and if there were none, the vial was placed in a freezer overnight. Solids that formed were isolated by filtration and dried.

Crash cooling: saturated solutions were prepared in various solvents or solvent systems at an elevated temperature and filtered through a 0.2-μm nylon filter into an open vial while still warm. The vial was covered and placed directly into a freezer. The presence or absence of solids was noted. Solids that formed were isolated by filtration and dried.

Antisolvent crystallization: solutions were prepared in various solvents at elevated temperature and filtered through a 0.2-μm nylon filter. Solid formation was induced by adding the filtered solution to an appropriate anti-solvent at a temperature below room temperature. The resulting solids were isolated by filtration and dried.

Slurrying: slurries were prepared by adding enough solids to a given solvent so that undissolved solids were present. The mixture was then agitated in a sealed vial at a chosen temperature. After time, the solids were isolated by filtration and dried.

Stress experiments: solids were stressed under different temperature and/or relative humidity (RH) environments for a measured time period. Specific RH values were achieved by placing the sample inside sealed chambers containing saturated salt solutions. Samples were analyzed by XRPD immediately after removal from the stress environment.

In addition to the starting material identified as form A, five additional solid forms were identified. Of the five additional forms, only one, form D, was confirmed to have an unsolvated structure, crystalline ezatiostat hydrochloride ansolvate. The other four forms were determined to be either hydrates, other solvates, or unstable forms.

Ansolvate Form D and its Properties

In one embodiment, this invention provides a crystalline ezatiostat salt ansolvate and, in particular, the hydrochloride ansolvate (crystalline form D). In another embodiment, this invention provides a composition comprising the crystalline ezatiostat hydrochloride ansolvate. Preferably, the crystalline form D is substantially free of a solvated polymorph of ezatiostat hydrochloride. “Substantially free” of a solvated polymorph of ezatiostat hydrochloride refers to a crystalline form D, which excludes solvated polymorph of ezatiostat hydrochloride to an extent that the form D crystals are suitable for human administration. In one embodiment, the crystalline form D contains up to about 5%, more preferably about 3%, and still more preferably about 1% of one or more solvated polymorph of ezatiostat hydrochloride. In one embodiment, the solvated polymorph is a form A, form B, or form E polymorph. As used herein, solvate includes hydrate form as well.

It is possible to attain the ansolvate form D with such high polymorphic purity due, in part, to the surprising stability of the ansolvate, and its resistance to conversion to a solvate form, even when stored at 40° C. and 75% RH without a desiccant for 6 months. See Table 1 below. In contrast, the solvate form E transforms almost entirely to form B crystals merely during tablet manufacture, which then transforms into a mixture of form B and the ansolvate form D within 3 months of storage at 40° C. and 75% RH without a desiccant. See Table 2 below. The solvate form A is also polymorphically unstable, converting into a mixture of forms A and D within 3 months of storage at 40° C. and 75% RH without a desiccant. See Table 3 below.

Not only was the ansolvate form D polymorphically stable, it was also more stable to chemical degradation compared to the polymorphs A, B, and E. See Tables 1-3 below in rows entitled “Total impurities”. Polymorphic form B, obtained from form E during tablet manufacture, was the most unstable, decomposing at more than double the rate of decomposition of the ansolvate form D. The stability of form D was enhanced even more, when stored in presence of a desiccant. Thus, in another embodiment, the present invention provides a crystalline ansolvate form D, which, when exposed to a temperature of about 25° C. for up to about 6 months in the presence of a desiccant, does not show substantial formation of an impurity. As used herein, “in the presence of a desiccant” refers to the desiccant being placed in a closed container with the ansolvate form D. The closed container, may be, but need not be sealed such that the air from the surrounding can not enter the closed container.

As used herein, “impurity” refers to one or more of: TLK 236, another polymorphic form of ezatiostat hydrochloride including without limitation form A, B, C, E, or F, and any other compound other than ezatiostat hydrochloride ansolvate, which may be identified by HPLC. TLK 236 is a monoester derived from the partial hydrolysis of ezatiostat where the phenyl glycine moiety remains esterified. “Does not show substantial formation of an impurity” refers to formation of only up to about 1.5% or more preferably up to about 1% of impurity.

The crystal form D is desirable from yet another standpoint, which is that, surprisingly, no other ansolvate form being identified upon screening, the ansolvate form D can not convert to another ansolvate polymorph upon storage or handling. And, as described above, ansolvate form D is stable with respect to a conversion to a solvate form, such as A, B, or E.

In another aspect, the present invention provides a method of storing comprising storing the crystalline ezatiostat hydrochloride ansolvate form D in the presence of a desiccant. In one embodiment, the desiccant is amorphous silicate. In another embodiment, the desiccant is Sorb-It silica gel. In one embodiment, the ansolvate form D is stored for up to 3 months, up to 6 months, up to 9 months, up to 1 year, up to 1.5 years, up to 2 years, or up to 3 years. In another embodiment, the ansolvate form D is stored at a temperature of up to about 25° C. In another embodiment, the ansolvate form D is stored at a temperature of up to about 40° C.

Furthermore, as part of a tablet, the ansolvate form D demonstrated higher aqueous dissolution rate than polymorphic form E (which converts to form B upon tableting) or B, when measured in 0.1 molar HCl, which is a convenient model for gastric fluid. Without being bound by theory, a higher dissolution rate relates to a higher amount of the active agent in the gastric fluid, which in turn relates to higher bioavailability of the active agent. A high bioavailability is desired, for example and without limitation, for reducing inter patient variability of drug exposure for a orally administered agent such as ezatiostat hydrochloride. So, for therapeutic use, the ansolvate form D is contemplated to be advantageous over form B or E. In one embodiment, the present invention provides a composition including the crystalline form D, which shows an aqueous solubility of at least about 5 mg/mL to about 20 mg/mL, about 10 mg/mL to about 15 mg/mL, about 5 mg/mL to about 15 mg/mL, or about 15 mg/mL to about 20 mg/mL. The aqueous solubility can be measured in a variety of aqueous solvents, including without limitation, water, 0.9% aqueous NaCl, 5% dextrose for injection, phosphate buffered saline, and generally aqueous solutions having a pH of less than about 5. Such solvents may include suitable buffers and other salts.

Preparation of Ansolvate Form D

In another aspect, this invention provides a method of preparing the solid crystalline ansolvate provided herein. In one embodiment, the method comprises slurrying ezatiostat hydrochloride in methyl tert-butyl ether at room temperature. In another embodiment, the method comprises slurrying ezatiostat hydrochloride in hexanes at about 60° C. In another embodiment, the method comprises heating ezatiostat hydrochloride monohydrate form A at a temperature from above about 155° C. up to less than the decomposition temperature and preferably to no more than about 180° C. for a period sufficient to convert the monohydrate to the ansolvate form D. Based on the present disclosure such transformations can be readily performed by the skilled artisan, for example, by monitoring DSC results.

In still another aspect, ezatiostat hydrochloride ansolvate is also obtained by dissolution of crude hydrated ezatiostat hydrochloride in about 5.6 times its weight of ethanol, heating to about (65-70)° C., filtering, seeding with a small quantity (e.g. about 2% by weight of the initial ezatiostat hydrochloride) of ezatiostat hydrochloride ansolvate, cooling to about 40° C., adding ethyl acetate in about 13.5 times the weight of the ezatiostat hydrochloride ansolvate, gradually cooling to about (20-25)° C. and then to (−5-0)° C., then filtering, washing with ethyl acetate, and drying.

Characterization of Crystalline Forms of Ezatiostat Hydrochloride

Crystalline ezatiostat hydrochloride ansolvate is characterized by its chemical composition, i.e. the presence of ezatiostat hydrochloride and the absence of water or other solvents of crystallization, and the crystalline nature of the material (the presence of an XRPD pattern characteristic of a crystalline, as opposed to amorphous, material). It may further conveniently be characterized by methods such as DSC, XRPD, and SS-NMR. It may also be characterized by other methods. These include analysis for water determination (typically by Karl Fischer analysis), where none or only a small quantity of water—significantly less than that which would be expected from a hydrate such as the monohydrate—should be found; and TG or TG-IR analysis, where none or only a small weight loss—significantly less than that which would be expected by the loss of a solvent of crystallization—would be found.

By DSC, crystalline ezatiostat hydrochloride ansolvate is characterized by an endothermic peak at (177±2)° C., which corresponds to melting of the crystalline ezatiostat hydrochloride ansolvate. If the crystalline ezatiostat hydrochloride ansolvate is free of other forms of ezatiostat hydrochloride, the DSC pattern will be characterized also by the substantial absence of thermal events at temperatures below the endothermic peak at (177±2)° C.; but the presence of minor quantities of other forms such as ezatiostat hydrochloride monohydrate will result in the presence of minor thermal events at lower temperatures. As used herein, “substantial absence of thermal events” refer to endotherms and exotherms related to melting and recrystallization.

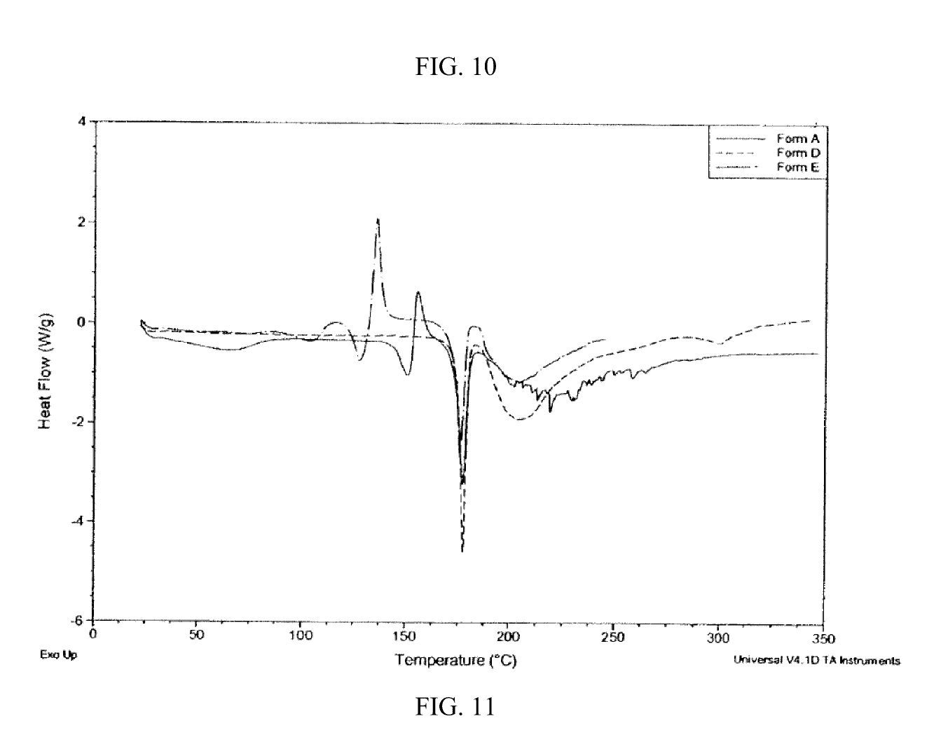

In one embodiment, this invention provides a crystalline ansolvate form D characterized by an endothermic peak at (177±2)° C. as measured by differential scanning calorimetry (DSC). In another embodiment, this invention provides a crystalline ansolvate form D characterized by substantial absence of thermal events at temperatures below the endothermic peak at (177±12)° C. as measured by differential scanning calorimetry. See, FIG. 10, which graphically illustrates a comparative DSC of forms A, D, and E, and demonstrates substantial absence of thermal events at temperatures below the endothermic peak at (177±12) CC for the crystalline ansolvate form D.

Under XRPD, crystalline ezatiostat hydrochloride ansolvate is characterized by a dominant zone with a rectangular planar (2-dimensional) unit cell with axial lengths of about 18.28 Å and 64.23 Å and an included angle of 90°; and systematic extinctions indicating that the planar cell has p2gg symmetry. Only two 3-dimensional space groups are consistent with the observed dominant zone cell and an ordered packing of a single diastereomer of a chiral molecule: these are orthorhombic space groups (P2₁2₁2₁ or P2₁2₁2₁) with approximate unit cell dimensions of a=64.23 Å, b=18.28 Å, c=short (P2₁2₁2), or a=short, b=18.28 Å, c=64.23 Å (P2₁2₁2₁). Note that permutations of the a and b axes are permissible for P2₁2₁2, and of all three axes for P2₁2₁2₁. The lowest-angle feature not related to the dominant zone is near 17.5° 2θ, indicating a short axis of about 5.1 Å (best match indexing solutions are consistent with about 5.08 Å, but there is insufficient peak resolution above 17° 2θ to definitively determine the length of the short axis and the space group). XRPD patterns will show peaks characteristic of this unit cell, as discussed further in the Examples below.

In another embodiment, this invention provides a crystalline ansolvate form D characterized by at least one X-ray powder diffraction peak (Cu Kα radiation) selected from 2.7°, 6.3°, 7.3°, 8.2°, 8.4°, 9.6°, 11.0°, and 12.7 °2θ (each ±0.2 °2θ). In another embodiment, this invention provides a crystalline ansolvate form D characterized by an X-ray powder diffraction peak (Cu Kα radiation) at (2.7±0.2) °2θ. In another embodiment, this invention provides a crystalline ansolvate form D characterized by an X-ray powder diffraction peak (Cu Ku radiation) at (6.3±0.2) °2θ. In another embodiment, this invention provides a crystalline ansolvate form D characterized by at least two X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ). In another embodiment, this invention provides a crystalline ansolvate form D characterized by at least three X-ray powder diffraction peaks (Cu Kα radiation) selected from 2.7°, 6.3°, 7.3°, 8.2°, 8.4°, 9.6°, 11.0°, and 12.7 °2θ (each ±0.2 °2θ). In another embodiment, this invention provides a crystalline ansolvate form D characterized by at least one X-ray powder diffraction peak (Cu Kα radiation) selected from 2.7°, 6.3°, 7.3°, 8.2°, 8.4°, 9.6°, 11.00, and 12.7 °2θ (each ±0.2 °2θ).

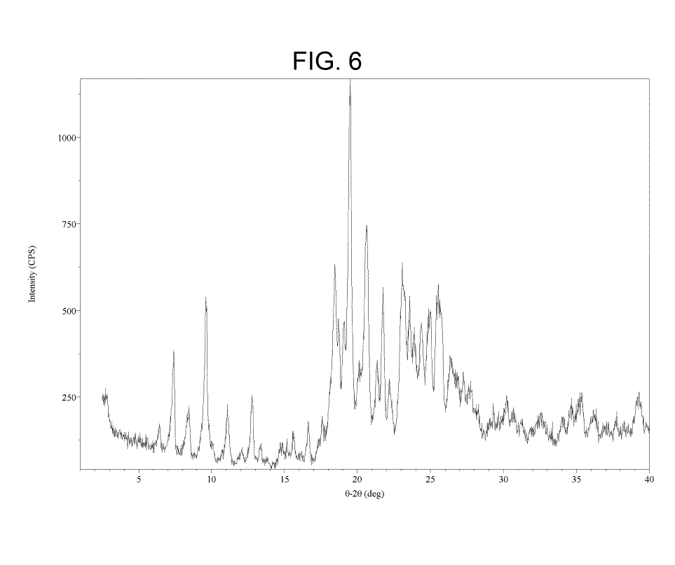

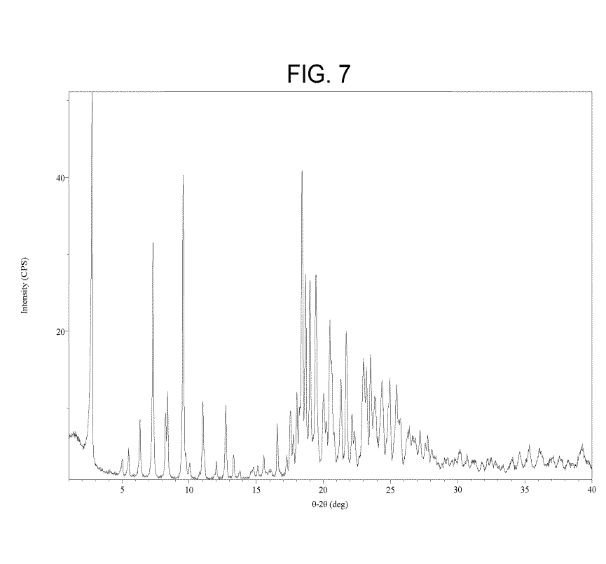

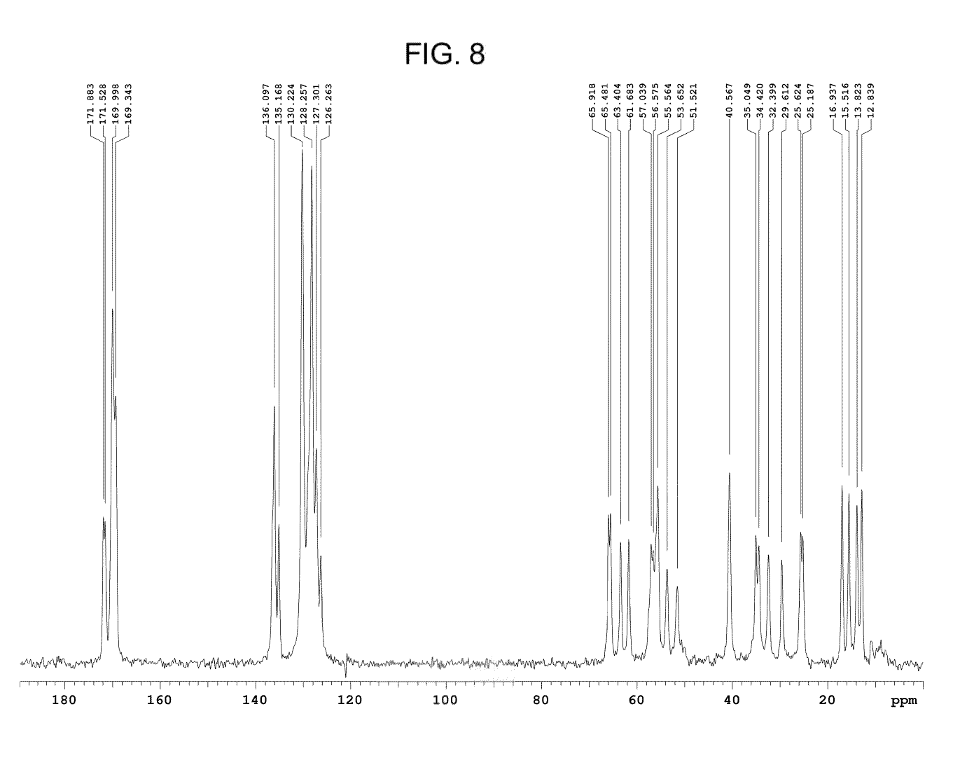

In another embodiment, this invention provides a crystalline ansolvate form D characterized by an X-ray powder diffraction pattern (Cu Kα radiation) substantially similar to that of FIG. 6 or FIG. 7. In another embodiment, this invention provides a crystalline ansolvate form D characterized by a solid-state ¹³C nuclear magnetic resonance spectrum substantially similar to that of FIG. 8.

Preparation and Characterization of Solvate Crystal Forms

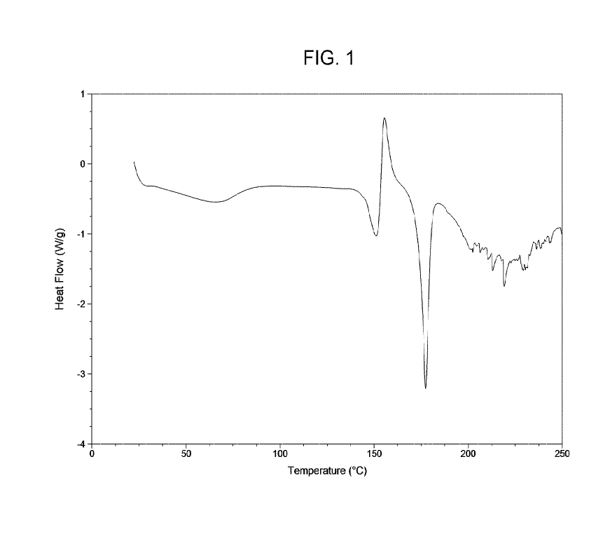

Form A was obtained from slurry experiments in ethyl acetate. Karl Fischer data indicated that form A contained approximately 1 mole of water for every mole of ezatiostat hydrochloride. However, thermal data indicated that the water could be lost easily. Stability and thermal data also indicated that form A readily converted to form B by increasing humidity, or to form D, when heated approximately at 153° C. DSC of ezatiostat hydrochloride monohydrate form A showed the pattern in FIG. 1, with a small broad endotherm at about 67° C., a larger and sharper endotherm with onset about 145° C. and peak at about 151° C. followed by an exotherm with peak at about 155° C. (corresponding to melting and recrystallization from the melt, as seen by hot stage microscopy) and a large sharp endothermic peak at about 177° C. (corresponding to melting, as seen by hot stage microscopy), followed by a broad endotherm at about (205-215)° C.

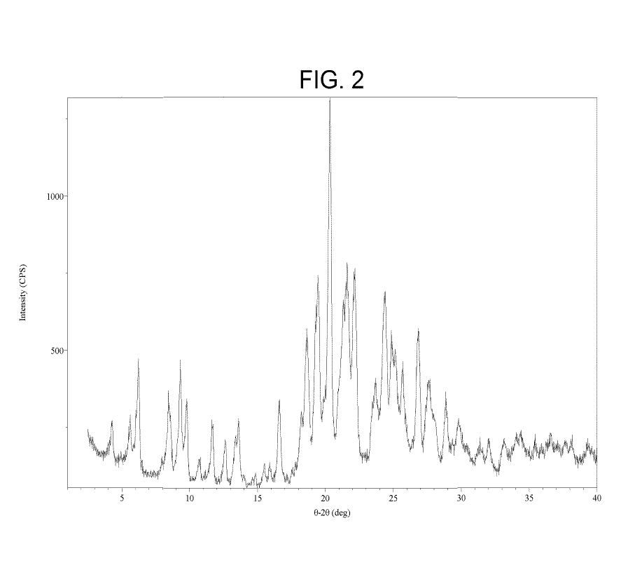

XRPD of ezatiostat hydrochloride monohydrate showed the pattern in FIG. 2. The nine largest peaks are at 5.6°, 6.2°, 9.3°, 13.6°, 18.6°, 20.3°, 21.3°, 24.4°, and 26.80° 2θ.

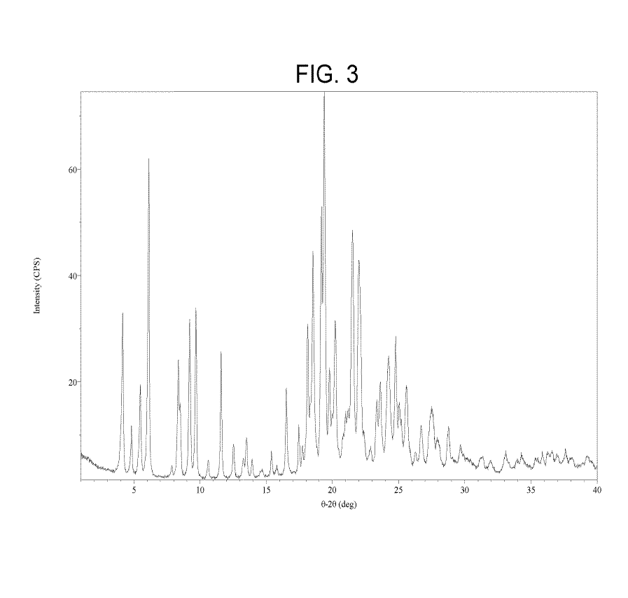

High-resolution XRPD of ezatiostat hydrochloride monohydrate showed the pattern in FIG. 3. The fifteen largest peaks are at 4.2°, 6.1°, 8.4°, 9.2°, 9.7°, 11.6°, 18.1°, 18.5°, 19.2°, 19.4°, 19.8°, 20.2°, 21.5°, 22.0°, and 24.8° 2θ. Minor differences from the pattern of FIG. 2 are seen, and these are considered likely to be due to preferred orientation and sample preparation effects. All of the low angle peaks are indexed using an oblique planar (2-dimensional) unit cell with axial lengths of about 21.3 Å and 29.1 Å and an included angle of 82.4° or 97.6°. This indicates that the XRPD pattern displays a “dominant zone” effect, implying the presence of one short and two long unit cell axes. The lowest-angle feature not related to the dominant zone is near 17.7° 2θ, indicating a short axis of about 5 Å; and there is insufficient peak resolution above 17° 2θ to determine the length of the short axis, the angles between the short axis and the longer axes, and the space group of the 3-dimensional unit cell.

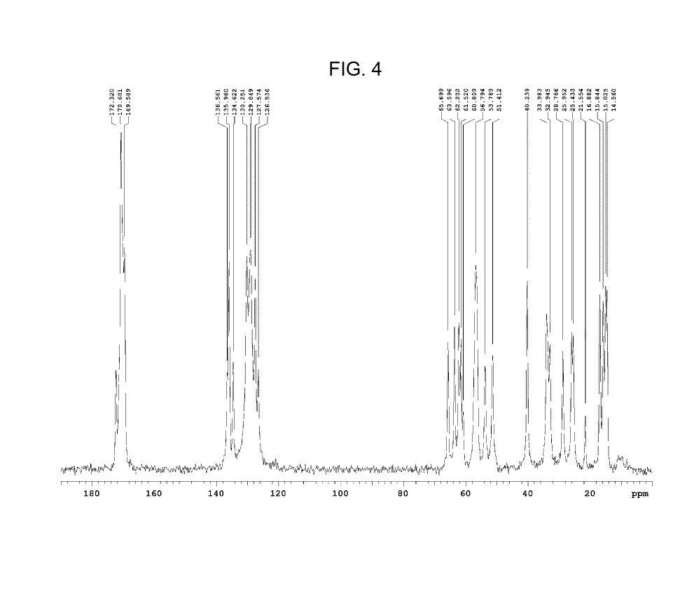

SS-NMR analysis of ezatiostat hydrochloride monohydrate showed the pattern in FIG. 4. Karl Fischer analysis of ezatiostat hydrochloride monohydrate showed a water content of 2.71% (3.08% expected for 1 mole of water). TG-IR analysis of ezatiostat hydrochloride monohydrate showed a weight loss between 30° C. and the maximum temperature of about 160° C. used in the analysis, where the volatile released below 110° C. was identified as water by IR.

Form B, which was prevalent in the polymorph screen experiments, was obtained after exposing form A to high relative humidity. A stress experiment indicated that form B could contain as many as 5 moles of water per mole of ezatiostat hydrochloride. Thermal data also suggested that form B converted to form D upon heating at 130° C.

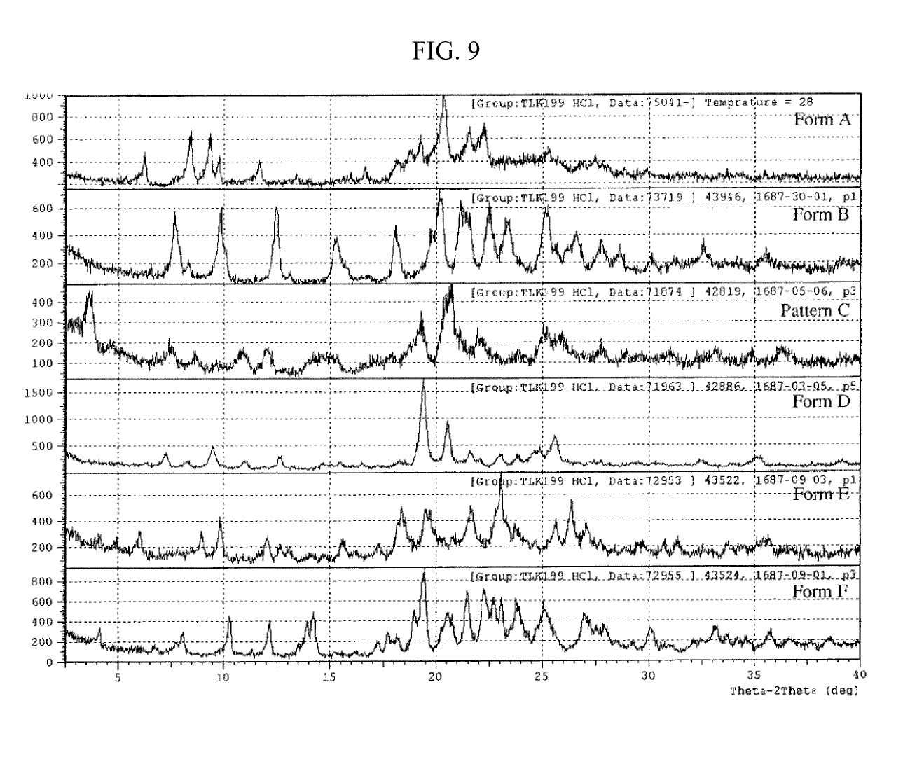

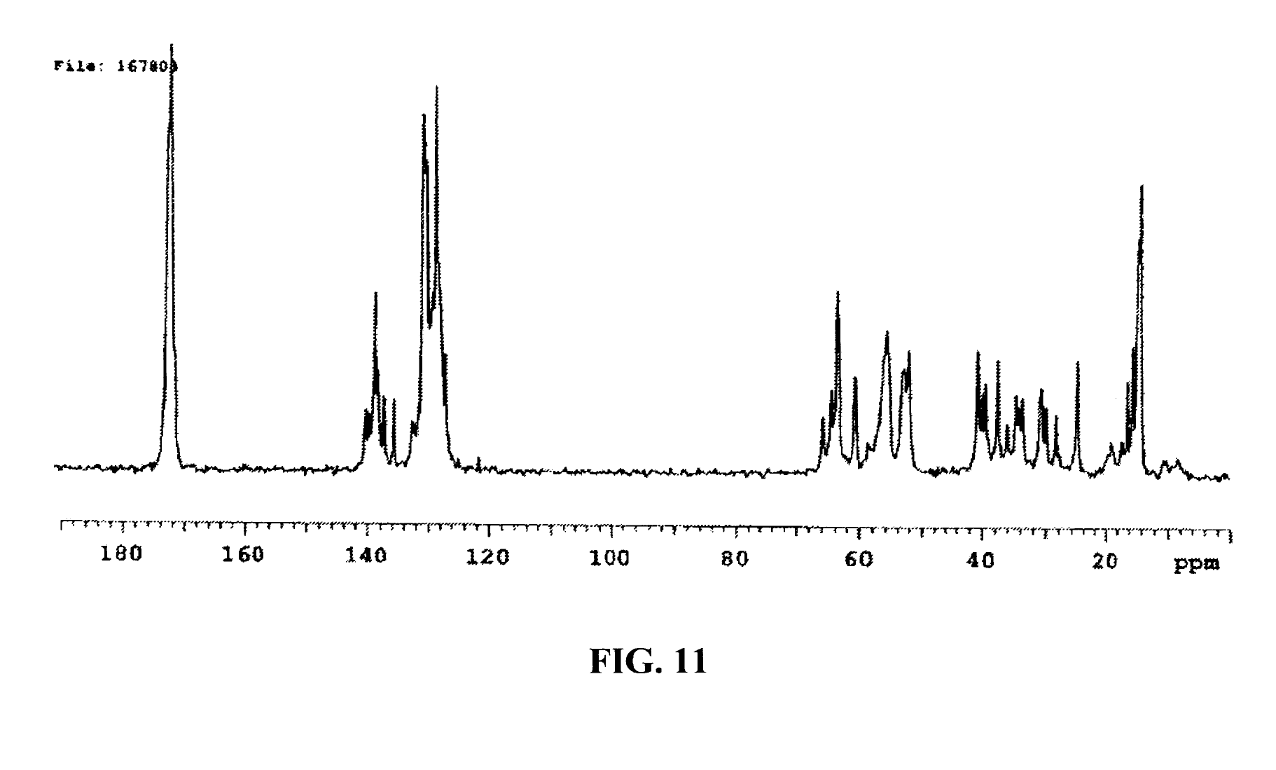

Form C, appeared to be an unstable polymorph, and was obtained from antisolvent crash precipitation experiments involving ethanol or methanol as the solubilizing solvent and ethyl acetate as the precipitating solvent. Due to its instability, this form could not be characterized further. Form E was obtained from cooling experiments in ethanol, and from antisolvent crash precipitation experiments involving ethanol and ethyl acetate. It was identified to be an ethanol solvate, based on TGIR weight loss experiments. DSC and SS-NMR of the form E polymorph are shown in FIGS. 10 and 11. The form F polymorph was obtained from slow cooling experiments in methanol. Based on TGIR weight loss experiments, it was identified to be a methanol solvate. The XRPD patterns of polymorphic forms A-F are shown in FIG. 9.

Treatment Methods

In another aspect, the present invention provides a method of treating myelodysplastic syndrome, severe chronic idiopathic neutropenia, leukemia or other cancers and conditions that involve cytopenia, chemotherapy induced neutropenia, or thrombocytopenia comprising administering a therapeutically effective amount of crystalline ezatiostat hydrochloride ansolvate (form D) to a patient in need of such treatment. Methods of therapeutic uses of ezatiostat are disclosed in U.S. Provisional Patent Applications 61/352,371, 61/352,373, and 61/352,374, each of which was filed on Jun. 7, 2010; the contents of which are incorporated herein by reference in their entirety.

### EXAMPLES

The following examples describe the preparation, characterization, and properties of ezatiostat hydrochloride ansolvate. Unless otherwise stated, all temperatures are in degrees Celcius (° C.) and the following abbreviations have the following definitions:

DSC Differential scanning calorimetry

GMP Good manufacturing practice

HPLC High performance liquid chromatography

NA Not applicable

ND Not determined

Q Percent dissolved per unit time

RH Relative humidity

RSD Residual standard deviation

RRT Relative retention time

SS-NMR Solid state nuclear magnetic resonance

TG-IR Thermogravimetric infra red analysis

XRPD X-ray powder diffraction

VT-XRPD Variable temperature X-ray powder diffraction

### Example 1

### Preparation of Ezatiostat Hydrochloride Ansolvate by Slurrying

Ezatiostat hydrochloride monohydrate was added to methyl tert-butyl ether at room temperature in excess, so that undissolved solids were present. The mixture was then agitated in a sealed vial at room temperature for 4 days, and the solids were then isolated by suction filtration. XRPD analysis of the solids established that the isolated solids were ezatiostat hydrochloride ansolvate.

Ezatiostat hydrochloride monohydrate was added to hexanes at 60° C. in excess, so that undissolved solids were present. The mixture was then agitated in a sealed vial at 60° C. for 4 days, and the solids were then isolated by suction filtration. XRPD analysis of the solids established that the isolated solids were ezatiostat hydrochloride ansolvate.

### Example 2

### Preparation of Crystalline Ezatiostat Hydrochloride Ansolvate by Heating

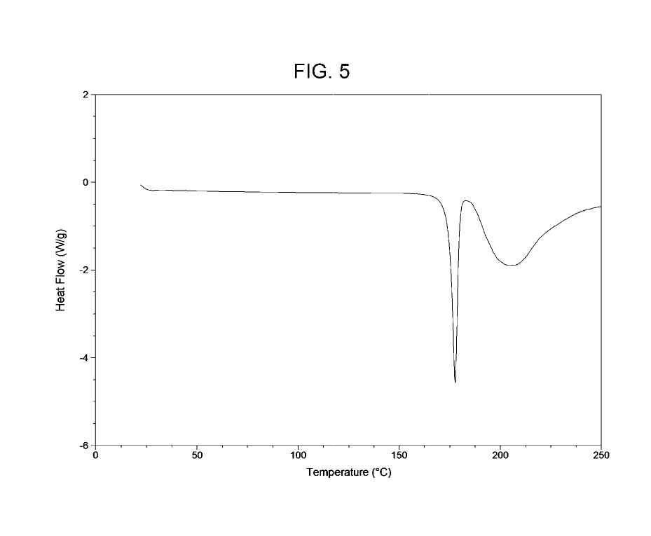

DSC of crystalline ezatiostat hydrochloride monohydrate showed the pattern in FIG. 1, as discussed in paragraph above. Hot stage microscopy showed an initial melt followed by a recrystallization at 153° C. and a final melt at 166° C. VT-XRPD, where XRPD patterns were obtained at 28° C., 90° C., and 160° C. during heating, and 28° C. after cooling of the formerly heated material, showed the presence of ezatiostat hydrochloride monohydrate at 28° C. and 90° C. during heating and of crystalline ezatiostat hydrochloride ansolvate at 160° C. and 28° C. after cooling of the formerly heated material. This confirmed that the transition at around 153/156° C. was a conversion of ezatiostat hydrochloride monohydrate form A to crystalline ezatiostat hydrochloride ansolvate form D and that the final DSC endothermic peak at about 177° C. (166° C. in the hot stage microscopy) was due to the melting of crystalline ezatiostat hydrochloride ansolvate. This was further confirmed by XRPD of the TG-IR material, where XRPD patterns obtained at room temperature both before and after heating to about 160° C. showed that the material before heating was form A and that the material after heating was form D ansolvate. DSC of crystalline ezatiostat hydrochloride ansolvate prepared by recrystallization showed the pattern in FIG. 5, with only the endothermic peak at about 177° C. followed by a broad endotherm at about (205-215)° C. Accordingly, the presence of the DSC endothermic peak at about 177° C., for example at (177±2)° C., when measured under the conditions described above, is considered characteristic of crystalline ezatiostat hydrochloride ansolvate, and the substantial absence of thermal events at temperatures below this is considered indicative of the absence of other forms of ezatiostat hydrochloride.

### Example 3

### Preparation of Crystalline Ezatiostat Hydrochloride Ansolvate by Crystallization

61.5 Kg crude ezatiostat hydrochloride was added to a reactor at room temperature, followed by 399 liter (L) ethanol, and this mixture was heated to 68° C. to completely dissolve the ezatiostat hydrochloride, filtered, then allowed to cool to 65° C. and checked for clarity and the absence of crystallization. About 1.3 Kg of ezatiostat hydrochloride ansolvate form D was suspended in 9 L of ethyl acetate, and about one-half of this suspension was added to the ethanol solution. The mixture was cooled to 63° C. and the second half of the suspension added to the mixture. The resulting mixture was cooled gradually to 45° C., 928 L ethyl acetate was added, and the mixture was cooled to 26° C. and held at about that temperature for about 5 hours, then cooled to −2° C. The mixture, containing crystalline ezatiostat hydrochloride ansolvate, was filtered, and the residue washed twice with 65 L of chilled (0-5° C.) ethyl acetate. The crystalline ezatiostat hydrochloride ansolvate was dried at 30° C. for 48 hours, then cooled to room temperature and sieved. Analysis of the material by DSC and XRPD confirmed its identity as crystalline ezatiostat hydrochloride ansolvate, and Karl Fischer analysis showed a water content of 0.1%.

XRPD of form D showed the pattern in FIG. 6. High-resolution XRPD of form D showed the pattern in FIG. 7. The major peaks are at 2.7°, 5.0°, 5.5°, 6.3°, 7.3°, 8.2°, 8.4°, 9.6°, 10.1°, 11.0°, 12.0°, 12.7°, 13.3°, 13.8°, 14.8°, 15.1°, 15.6°, 16.1°, 16.6°, 17.3°, 17.5°, 17.8°, 18.0°, 18.4°, 18.7°, 19.0°, 19.5°, 20.0°, 20.5°, 21.3°, 21.7°, 22.1°, 22.3°, 23.0°, 23.2°, 23.5°, 23.8°, 24.4°, 24.9°, 25.4°, 25.7°, 26.4°, 26.7°, 27.2°, 27.6°, 27.8°, 28.0°, and 29.3° 2θ. These peaks listed here at less than about 15° 2θ exhibit good separation from each other and are easily discernable even at lower resolution. Low angle peaks such as the peaks at 2.7°, 6.3°, 7.3°, 8.2°, 8.4°, 9.6°, 11.0°, and 12.7° 2θ are particularly useful in characterization of crystalline ezatiostat hydrochloride ansolvate; and at least one, preferably at least two, more preferably at least three of these peaks may be used. In particular, the peaks at 2.7° and 7.3° 2θ, especially the peak at 2.7° 2θ, may be considered characteristic of crystalline ezatiostat hydrochloride ansolvate.

SS-NMR analysis of crystalline ezatiostat hydrochloride ansolvate showed the pattern in FIG. 8, clearly distinguishable from that of ezatiostat hydrochloride monohydrate.

In summary, crystalline ezatiostat hydrochloride ansolvate form D is characterized by chemical composition, i.e. the presence of ezatiostat hydrochloride and the absence of water or other solvents of crystallization, and the crystalline nature of the material (the presence of an XRPD pattern characteristic of a crystalline, as opposed to amorphous, material). Additionally, the presence of the DSC endothermic peak at (177±12)° C. alone, or the presence of one or more of the low angle XRPD peaks (especially the peak at 2.7° 2θ, alone or with one or more of the other peaks below 15° 2θ, such as the peaks at 6.3°, 7.3°, 8.2°, 8.4°, 9.6°, 11.0°, and 12.7° 2θ, especially such as the peak at 7.3° 2θ and optionally one or more of the other peaks listed), preferably also in the absence of peaks indicative of ezatiostat hydrochloride monohydrate or other forms of ezatiostat hydrochloride, are considered characteristic of crystalline ezatiostat hydrochloride ansolvate. Also considered characteristic of crystalline ezatiostat hydrochloride ansolvate is XRPD patterns substantially the same as those in FIG. 6 or FIG. 7, when measured under the conditions described above.

### Example 4

### Polymorphic and Physicochemical Stability of Form D Ansolvate in the Absence of Desiccants

This example demonstrates the superior stability and solubility of the ansolvate form D compared to the solvate forms A, B, and E. Tablets of forms B, D, and E were made and stored at 40° C./75% RH without a desiccant for up to 6 months and the various properties of the tablets determined initially, and at 3 and 6 month intervals. As described above, form E converts to form B simply during tableting. The results are tabulated below.

TABLE 1

| API Polymorph | | | | |
|---|---|---|---|---|
| Form | | Polymorph Form D | | |
| Timepoint | | Initial | 3 Month | 6 Month |
| Description | White to off- | White round tablet | Off-white round tablet | Brown round tablet |
| | white round tablet | | | |
| Assay (HPLC) | 93.0-107.0% | 101.4 | 100.3 | 96.5 |
| | Label Claim | | | |
| Dissolution | Q = 70% of label | At 45 min, individual | At 45 min, individual | At 45 min, individual |
| | claim dissolved in | results: 37, 50, 32, 73, 54, | results: 95, 93, 96, 67, 98, | results: 68, 95, 73, 80, 71, |
| | 45 min | 57 | 81 | 100 |
| | | Mean = 51 | Mean = 88 | Mean = 81 |
| | | RSD% = 29.1 | RSD% = 13.6 | RSD% = 16.3 |
| Water Content | ≦5.0% | 0.9 | 0.8 | 0.8 |
| X-ray Diffraction | Report results | Polymorph D | Polymorph D | Polymorph D |
| Individual | RRT = 0.59/0.62 | ND | ND | 0.08 |
| Impurities | | | | |
| | RRT = 0.74 | 0.21 | 0.21 | 0.20 |
| | RRT = 0.80 | ND | ND | ND |
| | RRT = 0.81 | ND | ND | ND |
| | RRT = 0.83 | ND | 0.07 | 0.09 |
| | RRT = O.86 | ND | ND | ND |
| TLK236 | RRT =0.88 | 0.35 | 1.33 | 2.07 |
| RRT = 0.94 | ND | 0.07 | ND | |
| RRT = 0.96 | 0.18 | 0.19 | 0.23 | |
| RRT = 0.99 | ND | ND | ND | |
| Total impurities | | 0.7 | 1.9 | 2.7 |

TABLE 2

| API Polymorph Form | | Polymorph Form E | | |
|---|---|---|---|---|
| Timepoint | | Initial | 3 Month | 6 Month |
| Description | White to off-white round tablet | White round tablet | Off-white round tablet | Brown round tablet |
| Assay (HPLC) | 93.0-107.0% Label Claim | 93.7 | 90.3 | 84.6 |
| Dissolution | Q = 70% of label | At 45 min, individual | At 45 min, individual | At 45 min, individual |
| | claim dissolved | results: 47, 49, 43, 45, | results: 47, 31, 27, 30, | results: 17, 18, 18, 21 |
| | in 45 min | 47, 47 | 23, 42 | 26, 16 |
| | | Mean = 46 | Mean = 33 | Mean = 19 |
| | | RSD% = 4.3 | RSD% = 26.9 | RSD% = 19.4 |
| Water Content | ≦5.0% | 3.5 | 2.3 | 2.3 |
| X-ray Diffraction | Report results | Polymorph B | Polymorph B and D | Polyrmorph B and D |
| Individual Impurities | RRT = 0.59/0.62 | ND | 0.07 | 0.15 |
| | RRT = 0.74 | 0.38 | 0.42 | 0.51 |
| | RRT = 0.80 | ND | 0.16 | 0.41 |
| | RRT = 0.81 | ND | 0.14 | 0.17 |
| | RRT = 0.83 | 0.34 | 0.31 | 0.16 |
| | RRT = 0.86 | ND | 0.06 | ND |
| TLK236 | RRT = 0.88 | 0.42 | 3.45 | 4.66 |
| | RRT = 0.94 | ND | 0.08 | ND |
| | RRT = 0.96 | 0.20 | 0.19 | 0.24 |
| | RRT = 0.99 | 0.12 | 0.20 | ND |
| Total impurities | | 1.5 | 5.1 | 6.3 |

TABLE 3

| API Polymorph Form | | Polymorph Form A | | |
|---|---|---|---|---|
| Timepoint | | Initial | 3 Month | 6 Month |
| Description | White to off-white | White round tablet | Off-white round tablet | Off-white round tablet |
| | round tablet | | | |
| Assay (HPLC) | 93.0-107.0% Label | 97.2 | 94.1 | 91.5 |
| | Claim | | | |
| Dissolution | Q = 70% of label | At 45 min, individual | At 45 min, individual | At 45 min, individual |
| | claim dissolved in | results: 12, 12, 11, 12, | results: 88, 49, 64, 81, | results: 79, 83, 73, 80, |
| | 45 min | 12, 11 | 77, 83 | 25, 86 |
| | | Mean = 12 | Mean = 74 | Mean = 71 |
| | | RSD% = 4.0 | RSD% = 19.7 | RSD% = 32.5 |
| Water Content | ≦5.0% | 2.1 | 1.7 | 1.9 |
| X-ray Diffraction | Report results | Polymorph A and D | Polymorph A and D | Polymorph A and D |
| Individual Impurities | RRT = 0.59/0.62 | ND | ND | 0.07 |
| | RRT = 0.74 | 0.13 | 0.16 | 0.15 |
| | RRT = 0.80 | ND | 0.08 | 0.14 |
| | RRT = 0.81 | ND | 0.07 | 0.05 |
| | RRT = 0.83 | 0.46 | 0.10 | ND |
| | RRT = 0.86 | ND | ND | ND |
| TLK236 | RRT = 0.88 | 0.45 | 1.99 | 2.92 |
| | RRT = 0.94 | ND | 0.07 | ND |
| | RRT = 0.96 | 0.16 | 0.16 | 0.21 |
| | RRT = 0.99 | ND | 0.07 | ND |
| Total impurities | | 1.2 | 2.7 | 3.5 |

### Example 4

### Polymorphic and Physicochemical Stability of Form D Ansolvate in Presence of Desiccants

The stability of the ansolvate form D was further improved when stored in presence of a desiccant as demonstrated in this example. Tablets of ansolvate form D, were packaged with and without desiccant (Sorb-It Cannister, 1 gram). Fifty tablets were packaged in a round, white 1500 mL bottle with a screw cap over an induction seal. Impurities were assayed by HPLC. When stored at 25° C./60% RH with desiccant for 3 months, no increase in total impurities was observed. When stored at 40° C./75% RH with desiccant for 3 months, total impurities increased only by 0.3%. When stored at 40° C./75% RH without desiccant for 6 months, total impurities still increased only by 1.1%. As tabulated below, the presence of desiccant appears to further increase the stability of the ansolvate form D.

TABLE 4

| Timepoint | | Initial | 3 Month | 3 Month | 3 Month |
|---|---|---|---|---|---|
| Storage | | NA | 40° C./75% RH without | 25° C./60% RH with | 40° C./75% RH with |
| | | | dessicant | dessicant | dessicant |
| Description | White to off-white | White round tablet | Off-white round tablet | White round tablet | Off-white round tablet |
| | round tablet | | | | |
| Assay (HPLC) | 93.0-107.O% Label Claim | 101.4 | 100.3 | 99.7 | 100.8 |
| Dissolution | Q = 70% of label | At 45 min, individual | At 45 min, individual | At 45 min, individual | At 45 min, individual |
| | claim dissolved | | | | |
| | in 45 min | results: 37, 50, 32, 73, 54, | results: 95, 93, 96, 67, 98, | results: 36, 57, 43, 29, | results: 87, 52, 84, 97, |
| | | 57 | 81 | 53, 59 | 77, 59 |
| | | Mean = 51 | Mean = 88 | Mean = 46 | Mean = 76 |
| | | RSD% = 29.l | RSD% 12.0 | RSD% = 12.2 | RSD% = 17.1 |
| Water Content | ≦5.0% | 0.9 | 0.8 | 0.5 | 0.6 |
| X-ray | Report results | Polymorph D | Polymorph D | Polymorph D | Polymorph D |
| Diffraction | | | | | |
| Individual | RRT = 0.74/0.72 | 0.21 | 0.18 | 0.16 | 0.16 |
| Impurities | | | | | |
| | RRT = 0.83 | ND | 0.08 | ND | 0.09 |
| TLK236 | RRT = 0.88 | 0.35 | 1.3 | 0.34 | 0.53 |
| | RRT = 0.94 | ND | 0.07 | ND | 0.06 |
| | RRT = 0.96 | 0.18 | 0.18 | 0.19 | 0.19 |
| Total | | 0.7 | 1.8 | 0.7 | 1.0 |
| impurities | | | | | |

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. Crystalline ezatiostat hydrochloride ansolvate.

2. The crystalline ezatiostat hydrochloride ansolvate of claim 1, which is substantially free of a solvated polymorph of ezatiostat hydrochloride.

3. A composition comprising the crystalline ezatiostat hydrochloride ansolvate of claim 1.

4. The crystalline ezatiostat hydrochloride ansolvate of claim 1 characterized by at least one X-ray powder diffraction peak (Cu Kα radiation) selected from 2.7°, 6.3°, 7.3°, 8.2°, 8.4°, 9.6°, 11.0°, and 12.7 °2θ (each ±0.2 °2θ).

5. A method of preparing a solid crystalline ezatiostat hydrochloride ansolvate of claim 1 comprising slurrying ezatiostat hydrochloride in methyl tert-butyl ether at room temperature.

6. A method of preparing a solid crystalline ezatiostat hydrochloride ansolvate of claim 1 comprising slurrying ezatiostat hydrochloride in hexanes at about 60° C.

7. A method of preparing the crystalline ezatiostat hydrochloride ansolvate of claim 1 comprising heating ezatiostat hydrochloride monohydrate form A at a temperature from above about 155° C. up to about 180° C.

8. A method of storing comprising storing the crystalline ezatiostat hydrochloride ansolvate of claim 1 in the presence of a desiccant.

9. A method of treating myelodysplastic syndrome, severe chronic idiopathic neutropenia, leukemia or other cancers and conditions that involve cytopenia, chemotherapy induced neutropenia, or thrombocytopenia comprising administering a therapeutically effective amount of the crystalline ezatiostat hydrochloride ansolvate of claim 1 or the composition of claim 3 to a patient in need of such treatment.
